(19) United States
(12) Patent Application Publication
(10) Pub. No.: US 2022/0304911 A1
(43) Pub. Date: Sep. 29, 2022

(54) ORAL CARE PRODUCT WITH CANNABINOID COMPOUNDS

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/656,646

(22) Filed: Mar. 26, 2022

Related U.S. Application Data
(60) Provisional application No. 63/166,818, filed on Mar. 26, 2021.
(60) Provisional application No. 63/181,038, filed on Apr. 28, 2021.

(51) Int. Cl. A61K 8/49; A61K 8/34; A61K 8/21; A61Q 11/00

(52) U.S. Cl. CPC A61K 8/498; A61K 8/347; A61K 8/21; A61Q 11/00

(57) ABSTRACT

The present disclosure relates to compositions and methods for reducing, preventing, and/or treating oral conditions comprising the administration of one or more cannabinoid compounds to reduce Streptococcus mutans. The one or more cannabinoid compounds can include cannabigerol (“CBG”), cannabigerolic acid (“CBGA”), cannabichromene (“CBC”), cannabidivarinic acid (“CBDVA”), cannabidivarin (“CBDV”), cannabidiolic acid (“CBDA”), cannabidiol (“CBD”), cannabidiol ethyl (“CBD-C2”), cannabidibutol (“CBD-C4”), (+)-cannabidiol (“(+)-CBD”), cannabigerovarin (“CBGV”), cannabigerol butyl (“CBG-C4”), or cannabigerolic acid butyl (“CBGA-C4”). Methods of making and using the cannabinoid compounds are further disclosed.

### CROSS-REFERENCE TO RELATED APPLICATIONS

The present application claims the priority benefit of U.S. Provisional Patent App. Ser. No. 63/166,818, filed Mar. 26, 2021, and U.S. Provisional Patent App. Ser. No. 63/181,038, filed Apr. 28, 2021, each of which are hereby incorporated by reference herein.

### TECHNICAL FIELD

The present disclosure relates generally to the use of cannabinoid compounds in various oral conditions. Specifically, the described cannabinoid compounds are useful in mitigating microbial growth to promote oral health or in treatment of one or more oral disorders caused by bacterial infection.

### BACKGROUND

Streptococcus mutans (S. mutans) is a facultatively anaerobic, gram-positive coccus commonly found in the human oral cavity. It is a significant contributor to tooth decay. S. mutans is well documented as a microorganism living in the mouth that can adhere to plaque and cause dental caries. It has been shown that by reducing levels of S. mutans in the plaque, dental caries can be limited, if not prevented. Such bacteria can further cause bad breath, infections or other oral diseases or disorders. The persistence of local bacteria irritants stimulate fluid and cellular exudates, which in turn causes degeneration of the oral tissue resulting in gingivitis and periodontal disease. There is an unmet need for safe and effective oral care compositions that prevent or reduce growth of oral bacteria, including but not limited to S. mutans.

### SUMMARY

The present disclosure is directed to a composition for use as an oral care product to reduce, treat, prevent, or ameliorate growth of Streptococcus mutans (S. mutans), comprising one or more cannabinoid compounds. The one or more cannabinoid compounds can comprise cannabigerol (“CBG”), cannabigerolic acid (“CBGA”), cannabichromene (“CBC”), cannabidivarinic acid (“CBDVA”), cannabidivarin (“CBDV”), cannabidiolic acid (“CBDA”), cannabidiol (“CBD”), cannabidiol ethyl (“CBD-C2”), cannabidibutol (“CBD-C4”), (+)-cannabidiol (“(+)-CBD”), cannabigerovarin (“CBGV”), cannabigerol butyl (“CBG-C4”), or cannabigerolic acid butyl (“CBGA-C4”).

In some embodiments, the composition is an oral care formulation. In some embodiments, the composition is a toothpaste. In some embodiments, the composition is a mouthwash. In some embodiments, the composition is a chewable. In some embodiments, the composition is a wipe. In some embodiments, the composition is a rinse. In some embodiments, the composition is a powder. In some embodiments, the composition is a floss.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of the one or more cannabinoid compounds. Each of the one or more cannabinoid compounds (e.g., CBG, CBGA, CBC, CBDVA, CBDV, CBDA, CBD, CBD-C2, CBD-C4, (+)-CBD, CBGV, CBG-C4, or CBGA-C4) can be included at the provided concentration or collectively at the provided concentrations.

In some embodiments, the composition comprises one or more additional oral care active ingredients.

In some embodiments, the composition comprises one or more of cetylpyridinium chloride, stannous fluoride, essential oil, and fluoride ions.

In some embodiments, the composition can comprise fluoride ions.

In one aspect, the present disclosure is directed to a method of making an oral care product comprising adding one or more cannabinoid compounds comprising CBG, CBGA, CBC, CBDVA, CBDV, CBDA, CBD, CBD-C2, CBD-C4, (+)-CBD, CBGV, CBG-C4, or CBGA-C4 to an oral care formulation.

In one aspect, the present disclosure is directed to an oral care product made by adding one or more cannabinoid compounds comprising CBG, CBGA, CBC, CBDVA, CBDV, CBDA, CBD, CBD-C2, CBD-C4, (+)-CBD, CBGV, CBG-C4, or CBGA-C4 to an oral care formulation.

In one aspect, the present disclosure is directed to a method of prevent or treating an oral condition associated with S. mutans in a subject in need thereof, comprising administering an effective amount of the composition of the present disclosure to the subject.

In one aspect, the present disclosure is directed to a method of reducing growth of S. mutans bacteria, comprising contacting the bacteria with an effective amount of the composition of the present disclosure to the subject.

In one aspect, the present disclosure is directed to method of cleaning the oral cavity and/or teeth of a subject in need thereof, comprising administering an effective amount of the composition of the present disclosure to the subject,

In some embodiments, wherein the method prevents or reduces growth of S. mutans.

### DETAILED DESCRIPTION

### Definitions

As used throughout, ranges are used as shorthand for describing each and every value that it is within the range. Any value within the range can be selected as the terminus of the range.

As used herein, the words “preferred” and “preferably” refer to embodiments of the disclosure that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein, the term “about”, when applied to the value for a parameter of a composition or method of this disclosure, indicates that the calculation or the measurement of the value allows some slight imprecision without having a substantial effect on the chemical or physical attributes of the composition or the method. If, for some reason, the imprecision provided by “about” is not otherwise understood in the art with this ordinary meaning, then “about” as used herein indicates a possible variation of up to 5% in the value.

As referred to herein, compositional percentages are by weight of the total composition, unless otherwise specified.

As used herein, “ppm” (parts per million) refers to ppm by weight, unless otherwise indicated.

As referred to herein, all ratios refer to weight ratios, unless otherwise indicated.

As used herein “minimum inhibitory concentration” or “MIC” refers to the lowest concentration (in μg/mL) of an antibiotic that inhibits the growth of a given strain of bacteria.

As used herein, the term “treating” may refer to, for example, an improvement of one of more symptoms of a condition and/or a delay in disease progression. As used herein, the term “preventing” may refer to, for example, a delay in disease onset compared to, e.g., a population average.

As used herein, the term “subject” may refer to, for example, a patient diagnosed with or suspected of having an oral condition that may benefit from the administration of a composition described herein. The terms “subject” and “patient” are used interchangeably herein. In some embodiments, the subject is human. In some embodiments, the subject is a human adult, e.g., is over 18 years of age, about 18-30 years of age, about 30-40 years of age, about 40-45 years of age, about 45-50 years of age, about 50-55 years of age, about 55-60 years of age, about 60-65 years of age, about 65-70 years of age, about 70-75 years of age, about 75-80 years of age, about 80-85 years of age, about 85-90 years of age, or over 90 years of age.

The cannabinoid compound combinations as discussed herein and compositions comprising the same can be used to treat one or more oral conditions within the mouth. “Mouth” as used herein refers to the cavity bounded externally by the lips and internally by the pharynx that encloses the tongue, gums and teeth. Thus, the tissues of the mouth include the lips, tongue, gums, buccal tissue, palate and teeth. A single tissue, a plurality of tissues, a portion of one or more tissues, all or substantially all of the tissues of the mouth, or combinations of the foregoing, may be treated according to the disclosure.

As used herein, “treat” and variations thereof as used herein refers to cure, ameliorate, alleviate, inhibit, prevent, reduce the likelihood of, or reduce the severity of, a disease or condition, or of at least some of the symptoms or effects thereof. To treat a tissue of the mouth, the tissue is contacted with the composition effective at treating the condition. In some embodiments, an oral bacterial infection with S. mutans may be contacted with an oral care composition comprising one or more cannabinoid compounds comprising CBG, CBGA, CBC, CBDVA, CBDV, CBDA, CBD, CBD-C2, CBD-C4, (+)-CBD, CBGV, CBG-C4, or CBGA-C4.

### Compositions

The present disclosure describes use of cannabinoid compounds to prevent, reduce or treat various oral conditions.

In one aspect, the disclosure describes compositions useful in the prevention or reduction of growth of S. mutans. By preventing the growth of S. mutans in the oral cavity, there would be minimal to no S. mutans available to bind to the plaque and adhere to fissures within the tooth and begin the process of decay leading to dental caries. Accordingly, the present disclosure provides, in one aspect, compositions for use as oral care products, where the composition includes one or more cannabinoid compounds comprising CBG, CBGA, CBC, CBDVA, CBDV, CBDA, CBD, CBD-C2, CBD-C4, (+)-CBD, CBGV, CBG-C4, or CBGA-C4 thereof in an amount effective to prevent or reduce growth of S. mutans. The composition may specifically prevent or reduce growth of S. mutans.

As can be appreciated, controlling or inhibiting the growth of S. mutans in the oral cavity is difficult as known antiseptic agents and antibiotics have numerous shortcomings. For example, known antiseptic agents such as chlorhexidine cause discoloration of teeth, alteration in taste, supragingival calculus formation, bitter taste, and other side effects which discourage use. Antibiotics such as tetracyclines, macrolides, lincosamides, β-lactams, and nitroimidazoles have also been used to attempt to inhibit or control S. mutans. However, treatments with existing classes of antibiotics have side effects and suffer from overuse. Although research is ongoing to locate more effective antibiotics, there is an unmet need for an agent which can inhibit the growth of S. mutans.

It has specifically been discovered that CBG, CBGA, CBC, CBDVA, CBDV, CBDA, CBD, CBD-C2, CBD-C4, (+)-CBD, CBGV, CBG-C4, and CBGA-C4 each have a minimum inhibitory concentration (“MIC”) against S. mutans of less than 25 μg/mL. Such a MIC value means that these cannabinoid compounds are highly effective at inhibiting the growth of S. mutans. Thus, these cannabinoid compounds can be included in various oral care products to reduce, or eliminate, an infection caused by S. mutans, and can reduce dental caries, periodontal disease, and other oral conditions caused by S. mutans.

Other cannabinoid compounds were not found to be effective at inhibiting the growth of S. mutans when present at low concentrations. For example, cannabicyclol (“CBL”), cannabinol (“CBN”), CBD-C1, cannabigerovarinic acid (“CBGVA”), cannabichromenic acid (“CBCA”), cannabicyclolic acid (“CBLA”), and cannabinolic acid (“CBNA”) were found to have a MIC of greater than 25 μg/mL against S. mutans.

Prior to the present discovery, it was not appreciated which specific cannabinoid compounds could act to inhibit the growth of S. mutans. By selecting the cannabinoid compounds with the greatest effectiveness against S. mutans, the present disclosure provides improved methods of inhibiting the growth of S. mutans, and methods of preventing, or treating, dental caries, periodontal disease, and other oral disorders.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat oral conditions with specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally derived cannabinoid compounds offer benefits in regard to the treatment of periodontal disease and other oral disorders by inhibiting the growth of S. mutans not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of treating S. mutans that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally derived cannabinoid compounds are sufficiently pure and/or if any containments are sufficiently well understood.

Generally, oral care products that reduce, or eliminate the growth of S. mutans can include an effective concentration of one or more of CBG, CBGA, CBC, CBDVA, CBDV, CBDA, CBD, CBD-C2, CBD-C4, (+)-CBD, CBGV, CBG-C4, or CBGA-C4. In certain embodiments, each of the foregoing cannabinoid compounds can be included while in other embodiments, only a subset of such cannabinoid compounds can be included. In certain embodiments, only a single cannabinoid compound can be included.

The effective concentration of the one or more cannabinoid compounds can vary depending on factors such as the desired effect of treatment, the severity of the S. mutans infection, the severity of the periodontal disease or other oral condition, the duration of treatment, and the method of delivering the cannabinoid compounds to the subject. For example, in certain embodiments, a chewing gum can include a smaller concentration of the cannabinoid compounds because the gum will provide longer exposure of the cannabinoid compounds to the desired areas of the oral cavity than other oral care products such as a mouth wash.

In certain embodiments, the concentration of the cannabinoid compounds can be about equal to the MIC value of the cannabinoid compounds. In other embodiments, the concentration of the cannabinoid compounds can be greater than the MIC value such as in examples where oral contact is shorter in duration (e.g., a toothpaste or mouthwash). In certain embodiments, the cannabinoid compounds can be included in the oral care product at a concentration of about 200 μg per mL, or g, of the oral care product or less.

In certain embodiments, the compositions, products, articles, and methods described herein can be substantially or entirely free of cannabinoid compounds other than of CBG, CBGA, CBC, CBDVA, CBDV, CBDA, CBD, CBD-C2, CBD-C4, (+)-CBD, CBGV, CBG-C4, and CBGA-C4. For example, the compositions, products, articles, and methods can be substantially or entirely free of cannabicyclol (“CBL”), tetrahydrocannabivarin (“THCV”), cannabinol (“CBN”), CBD-C1, cannabigerovarinic acid (“CBGVA”), cannabichromenic acid (“CBCA”), cannabicyclolic acid (“CBLA”), or cannabinolic acid (“CBNA”). As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compounds, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the compositions, products, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds. In certain embodiments, it can be useful to use the compositions, products, articles, and methods described herein as part of routine oral health care to prophylactically treat against infection with S. mutans. In other embodiments, the oral health care products described herein can be used only by oral health professionals (e.g., dentists, oral surgeons, etc.) or under their supervision.

Generally, the cannabinoid compounds can be included in any known oral health care product. For example, the cannabinoid compounds can be included in toothpaste, mouthwash, gargles, solutions, drops, emulsions, suspensions, liquids, gels, lozenges, mints, chewable, wipes, powders, or floss. The oral health care products can be intended for use by consumers and patients and/or be intended for use by dental professionals (e.g., dental hygienists, dentists and oral surgeons).

In some variations, the composition is an oral care formulation, such as a toothpaste, mouthwash, chewable, wipe, rinse, powder, or floss. In another aspect, the disclosure provides methods of making an oral care product by adding one or more cannabinoid compounds comprising CBG, CBGA, CBC, CBDVA, CBDV, CBDA, CBD, CBD-C2, CBD-C4, (+)-CBD, CBGV, CBG-C4, CBGA-C4 and an oral care product made by such methods.

It should be understood that the detailed description and specific examples, whilst indicating embodiments of the disclosure, are intended for the purpose of illustration only and are not intended to limit the scope of the invention.

### Compositions for Treating Oral Bacteria and Associated Conditions

In some embodiments, the composition of the present disclosure comprises CBG, CBGA, CBC, CBDVA, CBDV, CBDA, CBD, CBD-C2, CBD-C4, (+)-CBD, CBGV, CBG-C4, CBGA-C4, or a combination thereof is a sufficient concentration to prevent or disrupt the growth of S. mutans in the mouth.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of one or more of CBG, CBGA, CBC, CBDVA, CBDV, CBDA, CBD, CBD-C2, CBD-C4, (+)-CBD, CBGV, CBG-C4, or CBGA-C4.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of one or more of CBG, CBGA, CBC, CBDVA, CBDV, CBDA, CBD, CBD-C2, CBD-C4, (+)-CBD, CBGV, CBG-C4, or CBGA-C4.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 1 mM. In some embodiments, the composition of the present disclosure comprises CBG, CBGA, CBC, CBDVA, CBDV, CBDA, CBD, CBD-C2, CBD-C4, (+)-CBD, CBGV, CBG-C4, or CBGA-C4 or a combination thereof at a concentration of between at least about 0.5 μM and 1 mM, between at least about 1 μM and 1 mM, between at least about 2 μM and 1 mM, between at least about 5 μM and 1 mM, between at least about 10 μM and 1 mM, between at least about 15 μM and 1 mM, between at least about 20 μM and 1 mM, between at least about 25 μM and 1 mM, between at least about 50 μM and 1 mM, between at least about 100 μM and 1 mM, between at least about 150 μM and 1 mM, between at least about 200 μM and 1 mM, between at least about 250 μM and 1 mM, between at least about 300 μM and 1 mM, between at least about 350 μM and 1 mM, between at least about 400 μM and 1 mM, between at least about 450 μM and 1 mM, or between at least about 500 μM and 1 mM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, CBGA-C4 at a concentration of between at least about 0.1 μM and 500 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 500 μM, between at least about 1 μM and 500 μM, between at least about 2 μM and 500 μM, between at least about 5 μM and 500 μM, between at least about 10 μM and 500 μM, between at least about 15 μM and 500 μM, between at least about 20 μM and 500 μM, between at least about 25 μM and 500 μM, between at least about 50 μM and 500 μM, between at least about 100 μM and 500 μM, between at least about 150 μM and 500 μM, between at least about 200 μM and 500 μM, between at least about 250 μM and 500 μM, between at least about 300 μM and 500 μM, between at least about 350 μM and 500 μM, between at least about 400 μM and 500 μM, or between at least about 450 μM and 500 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 250 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 250 μM, between at least about 1 μM and 250 μM, between at least about 2 μM and 250 μM, between at least about 5 μM and 250 μM, between at least about 10 μM and 250 μM, between at least about 15 μM and 250 μM, between at least about 20 μM and 250 μM, between at least about 25 μM and 250 μM, between at least about 50 μM and 250 μM, between at least about 100 μM and 250 μM, between at least about 150 μM and 250 μM, or between at least about 200 μM and 250 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 100 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 100 μM, between at least about 1 μM and 100 μM, between at least about 2 μM and 100 μM, between at least about 5 μM and 100 μM, between at least about 10 μM and 100 μM, between at least about 15 μM and 100 μM, between at least about 20 μM and 100 μM, between at least about 25 μM and 100 μM, or between at least about 50 μM and 100 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 75 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 75 μM, between at least about 1 μM and 75 μM, between at least about 2 μM and 75 μM, between at least about 5 μM and 75 μM, between at least about 10 μM and 75 μM, between at least about 15 μM and 75 μM, between at least about 20 μM and 100 μM, between at least about 25 μM and 75 μM, or between at least about 50 μM and 75 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 50 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 50 μM, between at least about 1 μM and 50 μM, between at least about 2 μM and 50 μM, between at least about 5 μM and 50 μM, between at least about 10 μM and 50 μM, between at least about 15 μM and 50 μM, between at least about 20 μM and 50 μM, or between at least about 25 μM and 50 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 25 μM. In some embodiments, the composition of the present disclosure one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 25 μM, between at least about 1 μM and 25 μM, between at least about 2 μM and 25 μM, between at least about 5 μM and 25 μM, between at least about 10 μM and 25 μM, between at least about 15 μM and 25 μM, or between at least about 20 μM and 25 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 20 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 20 μM, between at least about 1 μM and 20 μM, between at least about 2 μM and 20 μM, between at least about 5 μM and 20 μM, between at least about 10 μM and 20 μM, or between at least about 15 μM and 20 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 15 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 15 μM, between at least about 1 μM and 15 μM, between at least about 2 μM and 15 μM, between at least about 5 μM and 15 μM, between at least about 10 μM and 15 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 10 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 10 μM, between at least about 1 μM and 10 μM, between at least about 2 μM and 10 μM, between at least about 5 μM and 10 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 5 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 5 μM, between at least about 1 μM and 5 μM, between at least about 2 μM and 5 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 2 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 2 μM, between at least about 1 μM and 2 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 1 μM. In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.5 μM and 1 μM.

In some embodiments, the composition of the present disclosure comprises one or more of CBG, CBGA, CBC, CBDVA, CBDV, CBDA, CBD, CBD-C2, CBD-C4, (+)-CBD, CBGV, CBG-C4, or CBGA-C4 at a concentration of between at least about 0.1 μM and 0.5 μM.

In some embodiments, the composition of the present disclosure comprises a combination of one or more of CBG, CBGA, CBC, CBDVA, CBDV, CBDA, CBD, CBD-C2, CBD-C4, (+)-CBD, CBGV, CBG-C4, or CBGA-C4.

### Oral Care Compositions

A wide variety of different oral care compositions can be prepared utilizing one or more cannabinoid compounds of the present disclosure using materials and methods known in the art or which will be developed. It is within the skill in the art to choose appropriate ingredients and combinations of ingredients and to determine the specific amount of one or more cannabinoid compounds to include in a particular oral care composition, given the knowledge in the art and the guidance provided herein.

Oral care compositions of the present disclosure include washes, rinses, gargles, solutions, drops, emulsions, suspensions, liquids, pastes, gels, ointments, creams, sprays, powders, tablets, gums, lozenges, mints, films, patches, and tooth whitening compositions. Oral care compositions of the invention include compositions intended for use by consumers and patients and compositions intended for use by dental professionals (e.g., dental hygienists, dentists and oral surgeons).

One or more cannabinoid compounds can be incorporated into an oral care composition or device, such as a toothpaste, a tooth gel, powder, wipe, a mouthwash or rinse, or a dental floss. In some embodiments, one or more cannabinoid compounds may be contained in a separate oral care composition or device which will be used separately from other compositions and devices employed in the prophylactic oral care regimen. For instance, one or more cannabinoid compounds can be incorporated into a mouthwash or rinse, a gum, a lozenge or a chewable tablet.

In some embodiments, dentifrices include toothpastes, tooth gels, tooth powders and liquid dentifrices. Toothpastes and tooth gels generally include a dental abrasive, a surfactant, a thickening agent, a humectant, a flavoring agent, a sweetening agent, a coloring agent and water. Toothpastes and tooth gels may also include opacifying agents, anti-caries agents, anti-calculus agents, tooth whitening agents, and other optional ingredients. Liquid dentifrices may comprise water, ethanol, a humectant, a surfactant, a thickening agent, an abrasive, an anti-caries agent, a flavoring agent and a sweetening agent.

In some embodiments, gels include dentifrice gels, non-abrasive gels and subgingival gels. Non-abrasive gels and subgingival gels generally include a thickening agent, a humectant, a flavoring agent, a sweetening agent, a coloring agent, and water. Such gels may also include one or more anti-caries agents and/or anti-calculus agents.

In some embodiments, creams generally include a thickening agent, a humectant and a surfactant, and may include a flavoring agent, a sweetening agent, a coloring agent.

In some embodiments, ointments suitable for oral use generally include one or more of the following: fats, oils, waxes, parafins, silicones, plastibase, alcohols, water, humectants, surfactants, thickening agents, talc, bentonites, zinc oxide, aluminum compounds, preservatives, antiviral compounds, and other ingredients.

In some embodiments, mouthwashes, rinses, gargles and sprays generally include water, ethanol, and/or a humectant, and may also include a surfactant, a flavoring agent, a sweetening agent, and a coloring agent, and may include a thickening agent and one or more anti-caries agents and/or anti-calculus agents.

Oral care devices of the disclosure include materials (such as sutures and sponges), flosses, tapes, chips, strips, fibers, a toothpick or rubber tip, syringes, dental implants and dental appliances (such as trays and troughs that fit over and cover the teeth and, optionally, the periodontal tissue) having one or more cannabinoid compounds adhered to, absorbed into, bound to, attached to, entrapped in, enclosed in, coated onto, or otherwise incorporated into, them.

As one example, a compressed chewable tablet comprises a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including one or more cannabinoid compounds, are dry blended and compressed into a tablet.

For instance, teeth are commonly whitened by applying a tooth whitening composition to the teeth by means of a dental tray or trough. One or more cannabinoid compounds could be incorporated into the tooth whitening composition that is used in the tray or trough. Alternatively, a separate composition comprising one or more cannabinoid compounds could be applied to the teeth in a cleaned or different tray or trough after the application of the tooth whitening composition is completed. In a further alternative, a wash or rinse comprising one or more cannabinoid compounds could be used to rinse the mouth before and/or after the application of the tooth whitening composition. Another composition for applying a tooth whitening composition to the teeth is a flexible strip. One or more cannabinoid compounds according to the present disclosure could be incorporated into such strips.

Chewing gum compositions generally include a gum base, a flavoring agent and a sweetening agent. Suitable gum bases include jelutong, rubber, latex, chicle, and vinylite resins, desirably with conventional plasticizers or softeners. Plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. One or more cannabinoid compounds may be incorporated into a gum base by, e.g., stirring them into a warm gum base or coating them onto the outer surface of the gum base.

In some embodiments, additional ingredients may be added to the oral care composition of the present disclosure. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, pH-adjusting agents, and many others.

Humectants suitable for use in oral care compositions include edible polyhydric alcohols such as glycerol, sorbitol, xylitol, butylene glycol, polyethylene glycol, propylene glycol, mannitol, and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacettes (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phospine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetyltrimethylammonium bromide, diisobuytylphenoxyethyldimethylbenzylammonium chloride, coconut alkyltrimetylammonium nitrite, cetylpyridinium fluoride). Certain cationic surfactants can also act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopol®, starch, copolymers of lactide and glycolide monomers (the copolymer having an average molecular weight of about 1,000-120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphopsphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing.

Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

A pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), phosphate salts (such as potassium and sodium phosphate), and combinations of the foregoing as needed to achieve and maintain the desired pH.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others.

Methods of contacting tissues of the mouth with oral care compositions are well known in the art. Suitable methods include rinsing the tissue with a solution (e.g., a mouthwash, rinse, spray, liquid dentifrice, or other solution), brushing the teeth with a dentifrice (e.g., a toothpaste, tooth gel, or powder), applying a non-abrasive solution, gel, paste, cream or ointment directly to the tissue (with or without the use of an applicator), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip, a finger, or a dental tray or appliance which allows for immersion of the teeth and, optionally, the periodontal tissue in, e.g., a gel or solution. thereof. In some embodiments, oral care compositions further comprise, for instance, sutures can be used to close a surgical wound or a wound resulting from a tooth extraction, dental floss can be used to floss the teeth, etc. The treatment of the tissue can be prophylactic treatment. For instance, the tissue may be treated as part of a prophylactic oral care regimen.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising one or more cannabinoid compounds as discussed herein, the wound(s) caused by the surgery or tooth extraction could be closed with sutures having one or more cannabinoid compounds as discussed herein incorporated into them, and/or the mouth could be rinsed immediately after the surgery or tooth extraction, and/or at intervals thereafter, with a solution comprising one or more cannabinoid compounds as discussed herein.

A composition comprising one or more cannabinoid compounds as discussed herein can be used to treat a disease or condition of oral tissue. Diseases and conditions treatable according to the disclosure include those cause by one or more oral bacteria. Additionally, such oral bacteria are known to be the cause or be associated with one or more inflammatory disease and conditions, such as gingivitis and periodontitis. In some embodiments, the composition reduces a S. mutans infection which improves inflammation and/or a condition associated with oral inflammation (e.g. periodontal disease, gingivitis). In some embodiments, such conditions treated by oral care compositions comprising one or more cannabinoid compounds which reduces a S. mutans infection as discussed herein may include periimplantitis, periodontitis, oral mucositis (especially oral mucositis caused by chemotherapy in cancer treatment), and dental pain. In some embodiments, a toothpaste, toothpowder or mouthwash comprising one or more cannabinoid compounds as discussed herein, may be used in teeth brushing and/or rinsing at a therapeutically effective amount to treat one or more bacterial infection.

It is understood by those skilled in the art that the dosage amount of a one or more cannabinoid compounds as discussed herein needed to treat a tissue of a subject's mouth will vary with the particular the type of oral care composition employed, whether the treatment is prophylactic or for the treatment of a disease or condition, the identity of the disease or condition to be treated, the severity of the disease or condition, the duration of the treatment, the identify of any other drugs being administered, the age, size and species of the animal, and like factors known in the medical and veterinary arts.

### EXAMPLES

To determine the ability of the cannabinoid compounds described herein to reduce, or eliminate, S. mutans, a minimum inhibitory concentration (“MIC”) assay was performed. The assay was performed by Charles River Discovery Research Services UK Limited (Portishead, United Kingdom) in accordance with industry standards. The results are depicted in Table 1.

TABLE 1

| Cannabinoid | MIC (μg/mL) |
|---|---|
| CBG | 25 |
| CBGA | 25 |
| CBC | 25 |
| CBDVA | 25 |
| CBL | >200 |
| CBN | 50 |
| CBDV | 25 |
| CBDA | 25 |
| CBD | 25 |
| CBD-C1 | 100 |
| CBD-C2 | 25 |
| CBD-C4 | 25 |
| (+)-CBD | 25 |
| CBGV | 25 |
| CBGVA | >200 |
| CBG-C4 | 25 |
| CBGA-C4 | 12.5 |
| CBCA | >200 |
| CBLA | >200 |
| CBNA | >200 |

As used herein, all percentages (%) are percent by weight of the total composition, also expressed as weight/weight %, % (w/w), w/w, w/w % or simply %, unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. An oral care product to treat, prevent, or ameliorate growth of Streptococcus mutans (“S. mutans”), the oral care product comprising one or more cannabinoid compounds comprising cannabigerol (“CBG”), cannabigerolic acid (“CBGA”), cannabichromene (“CBC”), cannabidivarinic acid (“CBDVA”), cannabidivarin (“CBDV”), cannabidiolic acid (“CBDA”), cannabidiol (“CBD”), cannabidiol ethyl (“CBD-C2”), cannabidibutol (“CBD-C4”), (+)-cannabidiol (“(+)-CBD”), cannabigerovarin (“CBGV”), cannabigerol butyl (“CBG-C4”), or cannabigerolic acid butyl (“CBGA-C4”).

2. The oral care product of claim 1 is a toothpaste, a mouthwash, a chewable, a wipe, a rinse, a powder, or a floss.

3. The oral care product of claim 1, wherein the composition comprises about 0.01 μg/mL to about 1 mg/mL of the one or more cannabinoid compounds.

4. The oral care product of claim 1 further comprises one or more additional oral care active ingredients.

5. The oral care product of claim 4, wherein the one or more additional oral care active indigents comprise stannous fluoride, essential oil, and fluoride ions.

6. The oral care product of claim 4, wherein the one or more additional oral care active indigents comprise fluoride ions.

7. The oral care product of claim 1, wherein the one or more cannabinoid compounds have a MIC against S. mutans of about 25 μg per mL or less.

8. The oral care product of claim 1, wherein the one or more cannabinoid compounds have a MIC against S. mutans of about 12.5 μg per mL or less.

9. The oral care product of claim 1 is effective in treating periodontal disease.

10. A method of making an oral care product comprising adding one or more cannabinoid compounds to an oral care formulation, and
wherein the one or more cannabinoid compounds comprise cannabigerol (“CBG”), cannabigerolic acid (“CBGA”), cannabichromene (“CBC”), cannabidivarinic acid (“CBDVA”), cannabidivarin (“CBDV”), cannabidiolic acid (“CBDA”), cannabidiol (“CBD”), cannabidiol ethyl (“CBD-C2”), cannabidibutol (“CBD-C4”), (+)-cannabidiol (“(+)-CBD”), cannabigerovarin (“CBGV”), cannabigerol butyl (“CBG-C4”), or cannabigerolic acid butyl (“CBGA-C4”).

11. The method of claim 10, wherein the one or more cannabinoid compounds reduce the amount of Streptococcus mutans (“S. mutans”) in the oral cavity.

12. The method of claim 10, wherein the one or more cannabinoid compounds substantially eliminate S. mutans in the oral cavity.

13. The method of claim 10, wherein the oral care product comprises toothpaste, a mouthwash, a chewable, a wipe, a rinse, a powder, or a floss.

14. A method of reducing growth of Streptococcus mutans bacteria, comprising contacting the bacteria with an effective amount of one or more cannabinoid compounds; and
wherein the one or more cannabinoid compounds comprise cannabigerol (“CBG”), cannabigerolic acid (“CBGA”), cannabichromene (“CBC”), cannabidivarinic acid (“CBDVA”), cannabidivarin (“CBDV”), cannabidiolic acid (“CBDA”), cannabidiol (“CBD”), cannabidiol ethyl (“CBD-C2”), cannabidibutol (“CBD-C4”), (+)-cannabidiol (“(+)-CBD”), cannabigerovarin (“CBGV”), cannabigerol butyl (“CBG-C4”), or cannabigerolic acid butyl (“CBGA-C4”).

15. The method of claim 14, wherein the one or more cannabinoid compounds reduce the amount of S. mutans in the oral cavity.

16. The method of claim 14, wherein the one or more cannabinoid compounds substantially eliminate S. mutans in the oral cavity.

17. The method of claim 14, wherein the one or more cannabinoid compounds are applied to the oral cavity using an oral care composition.

18. The method according of claim 14, wherein the one or more cannabinoid compounds are applied to the oral cavity using an oral care product.

19. The method of claim 14, wherein the one or more cannabinoid compounds are prophylactically applied.
